(19) United States
(12) Patent Application Publication
(10) Pub. No.: US 2021/0128762 A1
(43) Pub. Date: May 6, 2021

(54) SYSTEMS, METHODS, AND APPARATUS FOR DEVICE SANITIZATION

(71) Applicant: PHONESOAP LLC, Lehi, UT (US)

(72) Inventors: Wesley David LaPorte, Provo, UT (US); Daniel Harrison Barnes, Lehi, UT (US); Preston Evan Price, Cottonwood Heights, UT (US); Christian Evan Graff, Provo, UT (US)

(21) Appl. No.: 17/087,373

(22) Filed: Nov. 2, 2020

Related U.S. Application Data
(60) Provisional application No. 62/929,772, filed on Nov. 1, 2019.

(51) Int. Cl. A61L 2/08; A61L 2/24; A61L 2/26

(52) U.S. Cl. CPC A61L 2/084; A61L 2/24; A61L 2202/14; A61L 2202/11; A61L 2202/122; A61L 2/26

(57) ABSTRACT

A sanitization device comprises primary emitters configured to emit sanitizing electro-optical (EO) radiation into an interior compartment. A support member is configured to hold and/or secure a target object within the interior compartment of a sanitization device. An end portion of the support member may extend into a secondary compartment comprising a secondary emitter configured to produce external stimulus adapted to excite and recover primary emitters from dormancy. The support member is configured to transmit EO radiation produced by the secondary emitter into the interior compartment to thereby prevent and/or recover from dormancy conditions of the primary emitters. Detection circuitry can detect activation failures of the primary emitters. Thermal management circuitry can limit certain critical failures of a sanitization device that result from activation failures of primary emitters.

### CROSS-REFERENCES TO RELATED APPLICATIONS

This application claims benefit of priority under 35 U.S.C. § 119 to U.S. Provisional Patent Application No. 62/929,772 titled SYSTEMS, METHODS, AND APPARATUS FOR DEVICE SANITIZATION and filed Nov. 1, 2019, which is hereby incorporated herein by reference in its entirety.

### TECHNICAL FIELD

The disclosure relates to apparatus, systems, methods, and apparatus for device sanitization.

### BACKGROUND

Portable devices, such as mobile phones, smart phones, personal digital assistants, and/or the like are increasingly ubiquitous. Portable devices can, however, attract and harbor potentially harmful contaminants and/or organisms, such as microbes, pathogens, viruses, bacteria and/or the like and, as such, may become vectors for disease and/or infection. Systems, methods, and apparatus for sanitizing portable devices may be desirable.

### BRIEF DESCRIPTION OF THE DRAWINGS

This disclosure includes and references the accompanying drawings, which provide a more particular description of the embodiments disclosed herein. The disclosure, however, is not limited to the particular embodiments depicted in the figures. The teachings of the disclosure may be utilized and/or adapted to other embodiments, and/or changes may be made to the disclosed embodiments, without departing from the scope of the disclosure.

FIGS. 1A and 1B are schematic block diagrams of embodiments of a sanitization device, according to the present disclosure.

FIGS. 2A-2D are schematic block diagrams of further embodiments of a sanitization device, according to the present disclosure.

FIGS. 3A-3G illustrate further embodiments of a sanitization device, according to the present disclosure.

FIGS. 4A-4F illustrate further embodiments of a sanitization device, according to the present disclosure.

FIGS. 5A-5C illustrate further embodiments of a sanitization device, according to the present disclosure.

### DETAILED DESCRIPTION

In a time of ongoing world-wide pandemic, concerns about contamination and spread of harmful organisms, such as the novel coronavirus COVID-19, are heightened. Portable devices are increasingly ubiquitous and notorious for attracting and harboring such harmful organisms. Disinfecting chemicals are known, but can cause damage to the surfaces of portable devices. UV light and other radiation can provide safe and reliable disinfecting qualities. Disclosed herein are systems, methods, and apparatus for sanitizing portable devices and improvements thereof.

Some sources of sanitizing radiation, when unused fora period of time, can drift into a state of dormancy or otherwise become inactive because of a low electron state, which limits proper operation. A stimulus can excite electrons in such sources of sanitizing radiation in dormancy to enable a recovery the from the inactive or dormant state. Disclosed herein are systems, methods, and apparatus for sanitizing portable devices that include a stimulus source to provide recovery radiation.

Detecting when to activate a stimulus source presents a challenge. A sanitizing radiation source can be in a dormant state, but it can also “burn out” or wear out and cease to be operational. Disclosed herein are systems, methods, and apparatus for sanitizing portable devices that include bulb operation detection circuitry.

When a sanitizing radiation source malfunctions or otherwise ceased to operate properly, electronics in the sanitizing device can heat up and create a risk of damage to the sanitizing device. Disclosed herein are systems, methods, and apparatus for sanitizing devices that include thermal management to prevent critical failure.

During a sanitization by a sanitizing device according to the present disclosure, a portable device is positioned in the sanitizing device and a period of time is needed to allow sanitizing radiation to disinfect the device. During this period of time, a user of the sanitizing device (e.g., an owner of a portable device) may want to walk away from the sanitizing device to perform some other task in the meantime, which leaves the sanitizing device and portable device therein unattended. The unattended sanitizing device and potentially valuable portable device therein presents a temptation of theft or more simply an undesired movement of the sanitizing device (e.g., in a closet or drawer by someone tidying up). Disclosed herein are systems, methods, and apparatus for sanitizing portable devices that include anti-movement and/or anti-theft features to provide notification of when a sanitizing device is unplugged and/or moved.

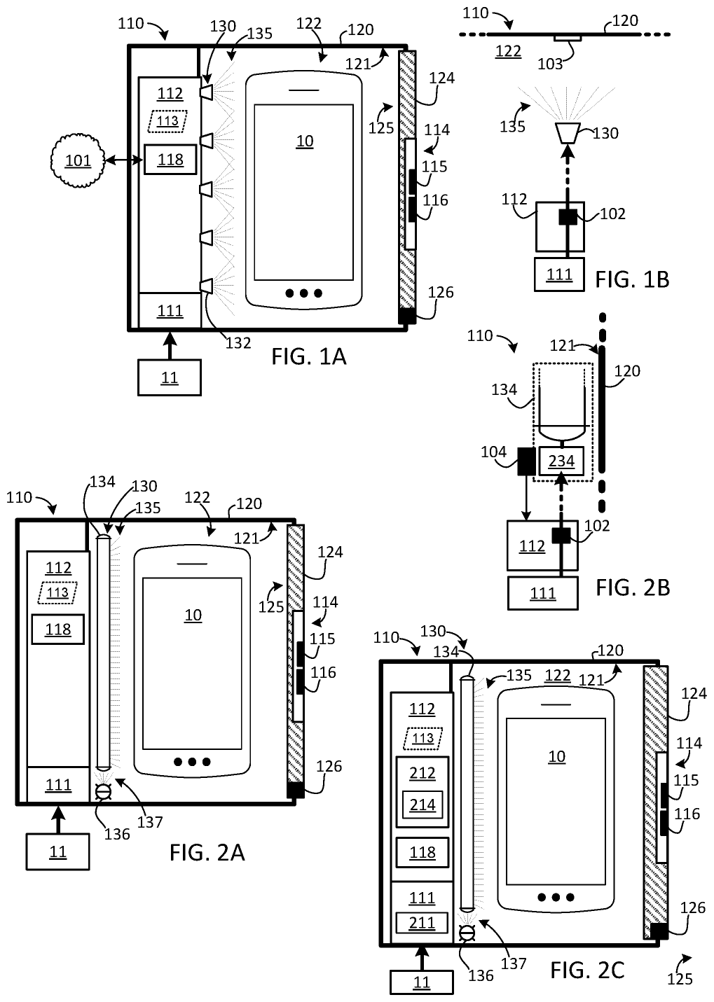

FIG. 1A is a schematic block diagram of one embodiment of a sanitization device 110, as disclosed herein (e.g., first embodiment). In the embodiment of FIG. 1A, the sanitization device 110 comprises an enclosure 120 configured to receive a target object 10 within an interior thereof (an interior compartment 122 defining an inner cavity or region). The target object 10 may comprise any suitable object including, but not limited to: a portable device, a computing device, an educational device, a calculator, a portable computing device, a communication device, a mobile communication device, a smart phone, a portable notation device, a portable media device, an image capture device, a video capture device, an audio capture device, a portable computing device, a tablet computing device, a laptop computer, a notebook computer, an electronic reading device, a personal digital assistant (PDA), a palmtop computer, a handheld computer, a pen computer, an ultra-mobile personal computer, a pager, a portable navigation device, a personal navigation assistant (e.g., portable GPS unit), a tool, a diagnostic device, a portable examination device (e.g., a stethoscope, reflex hammer, pulse oximetry device, and/or the like), a portable treatment device, a portable communication device, a phone, a wireless headset, a calculator, and/or the like.

The sanitization device 110 may be configured to sanitize target objects 10 by use of sanitizing electro-optical (EO) radiation. As used herein, sanitizing EO radiation or sanitizing light refers to any suitable wavelength and/or type of EO radiation or light capable of sanitizing a target object 10 (and/or a surface thereof), which may include, but is not limited to: type-C ultraviolet radiation (UV-C) comprising wavelengths between 280 and 100 nm; type B ultraviolet radiation (e.g., UV-B); middle ultraviolet (MUV) radiation; far ultraviolet radiation (FUV); ionizing EO radiation; non-ionizing EO radiation; a combination of a plurality of EO radiation wavelengths; and/or the like. The sanitizing device 110 may be configured to produce sanitizing EO radiation by use of one or more sanitizing EO radiation components 130. As used herein, a sanitizing EO radiation (SEOR) component 130 refers to any suitable means for generating, emitting, and/or otherwise producing sanitizing EO radiation 135 including, but not limited to: a light emitting diode (LED), an electric arc discharge device, a gas-discharge lamp, a fluorescent lamp, a compact fluorescent lamp (CFL), a Cold Cathode Fluorescence Lamp (CCFL), a laser, an incandescent lamp, and/or the like. In the FIG. 1A embodiment, the SEOR component(s) 130 of the sanitization device 110 comprise LED(s) 132.

An interior surface 121 of the enclosure 120 and/or interior compartment 122 may be configured to reflect and/or transmit sanitizing EO radiation 135. The interior surface 121 may be configured to reflect sanitizing EO radiation 135 within the interior compartment 122. The interior surface 121 may be comprised of material(s) configured to reflect sanitizing EO radiation 135, may comprise a coating configured to reflect sanitizing EO radiation 135, and/or the like. The interior surface 121 may be configured and/or shaped such that the sanitizing EO radiation 135 emitted by the SEOR component(s) 130 irradiates substantially all of the target object 10 (and/or substantially all of the exposed surface(s) of the target object 10).

The sanitization device 110 may comprise an access component 124 configured to control physical access to the interior compartment 122 (or inner cavity) of the enclosure. The access component 124 may comprise any suitable means for controlling physical access to the interior compartment 122 of the enclosure 120 including, but not limited to: a door, a hatch, a panel, a plate, a cover, a lid, a flap, a clamshell, a seal, a porthole, and/or the like. An inner surface 125 of the access component 124 may comprise a portion of the inner surface 121 of the enclosure 120. The inner surface 125 of the access component 124 may be configured to reflect and/or transmit sanitizing EO radiation 135 within the interior compartment 122, as disclosed herein.

The access component 124 may comprise and/or be configured to transition between an open configuration and closed configuration. As used herein, an open configuration of the sanitization device 110 (and/or access component 124 thereof) refers to a configuration in which the interior compartment 122 of the enclosure 120 is accessible from an exterior of the enclosure 120. A closed configuration of the sanitization device 110 (and/or access component 124 thereof) refers to a configuration in which the interior compartment 122 of the enclosure 120 is inaccessible, closed, secured, and/or sealed. The access component 124 may be configured to seal the enclosure 120 in the closed configuration (e.g., the closed configuration may comprise a sealed configuration). As used herein, sealing the enclosure 120 refers to enclosing the interior compartment 122 such that sanitizing EO radiation 135 emitted within the enclosure 120 is substantially contained, blocked and/or otherwise prevented from escaping from the enclosure 120 and/or sanitization device 110. The access component 124 may further comprise a secured or locked configuration. As used herein, a secured configuration or locked configuration refers to a configuration in which the sanitization device 110 (and/or access component 124 thereof) is secured and/or locked in the closed configuration (e.g., is secured and/or locked from transitioning out of the closed configuration).

In some embodiments, the sanitization device 110 further comprises a closure component 126 configured to selectively lock and/or secure the access component 124 in the closed configuration (e.g., selectively place the sanitization device 110 and/or access component 124 thereof in the secured and/or locked configuration). The closure component 126 may comprise any suitable means for securing and/or locking the access component 124 in the closed configuration including, but not limited to: a latch, a fastener, a fastening device, a mechanical fastening device, an electronic fastening device, a security device, a lock, a locking mechanism, a mechanical locking mechanism, a motorized locking mechanism, a magnetic locking mechanism, a time lock (a locking mechanism configured to remain in a locked configuration for a specified time period and/or release the lock on expiration of the specified time period), and/or the like.

The closure component 126 may be further configured to determine a current state of the access component 124, which may comprise determining whether the access component 124 is one of the: open configuration, closed configuration, sealed configuration, locked configuration, secured configuration, and/or the like. The closure component 126 may comprise and/or be coupled to means for determining the current state of the access component 124 (detection means), which may include, but are not limited to a: switch, contact switch, conductive switch, magnetic switch, capacitive switch, resistive switch, inductive switch, detector, contact detector, conductive detector, magnetic detector, capacitive detector, resistive detector, inductive detector, latch, and/or the like. In some embodiments, the closure component 126 may comprise a plurality of redundant locking and/or detection means.

The sanitization device 110 may further comprise a power module 111, which may be configured to supply power to the sanitization device 110 (and/or components thereof). The power module 111 may be further configured to supply power to the target object 10 (supply power to a smartphone disposed within the enclosure 120, as illustrated in FIG. 1A). The power module 111 may be configured to receive electrical power from an external power source 11. Alternatively, or in addition, the power module 111 may comprise an internal power source, such as power storage means (e.g., a battery, capacitor, super capacitor), a piezoelectric power source, a fuel cell power source, and/or the like (not shown in FIG. 1A to avoid obscuring details of the illustrated embodiments).

The sanitization device 110 may further comprise control componentry 112, which may be configured to manage, regulate, monitor, and/or otherwise control the sanitization device 110 and/or operation thereof. The control componentry 112 may comprise any suitable management, regulation, monitoring, and/or control means including, but not limited to: circuitry, control circuitry, a microcontroller, a microcontroller unit (MCU), a programmable logic controller (PLC), a control board, a processor, memory, random access memory (RAM), non-transitory storage, network interface components, and/or the like. Portions of the control componentry 112 may be embodied as computer-readable instructions stored on non-transitory storage (not shown in FIG. 1A to avoid obscuring details of the illustrated embodiments). Alternatively, or in addition, portions of the control componentry 112 may be embodied as hardware components, as disclosed herein such as circuitry, an Application-Specific Integrated Circuit (ASIC), a package, a die, a chip, a system-on-chip (Soc), a printed circuit board (PCB), and/or the like.

In some embodiments, the control componentry 112 may be operatively and/or communicatively coupled to the access component 124 (and/or closure component 126 thereof). The control componentry 112 may, therefore, be configured to determine and/or monitor the state of the access component 124 (e.g., whether the access component 124 is currently open, closed, locked, and/or the like). The control componentry 112 may be further configured to selectively lock and/or unlock the sanitization device 110 (e.g., by use of the access control component 124 and/or closure component 126, as disclosed herein).

The control componentry 112 may be operatively and/or communicatively coupled to the SEOR components 130 (e.g., the LED(s) 132). The control componentry 112 may be configured to selectively activate respective SEOR components 130. As used herein, activating an SEOR component 130 refers to one or more of: coupling the SEOR component 130 to power, configuring the SEOR component 130 to produce sanitizing EO radiation 135, and/or the like. The control componentry 112 may be further configured to monitor a status of respective SEOR components 130. Monitoring the status of an SEOR component 130 may comprise measuring, tracking, capturing, aggregating, determining and/or maintaining characteristics and/or diagnostic information pertaining to the SEOR component 130, which may include, but is not limited to: whether the SEOR component 130 is functional (e.g., whether the SEOR component 130 is capable of being activated and/or generating sanitizing EO radiation), a current state of the SEOR component 130 (e.g., whether the SEOR component 130 is currently active, coupled to power and/or configured to emit sanitizing EO radiation 135), characteristics of sanitizing EO radiation 135 produced by the SEOR component 130, power input to the SEOR component 130, power output of the SEOR component 130, efficiency of the SEOR component 130, electrical characteristics of the SEOR component 130 (e.g., current flow through the SEOR component 130, voltage drop across the SEOR component 130, impedance of the SEOR component 130, and/or the like), physical characteristics of the SEOR component 130 (e.g., temperature of the SEOR component 130), the time for which the SEOR component 130 has been active during a current sanitization operation or cycle, a cumulative time for which the SEOR component 130 has been active (e.g., over the lifetime of the sanitization device 110), and/or the like.

FIG. 1B is a schematic block diagram illustrating further embodiments of the disclosed sanitization device 110. As disclosed above, the control componentry 112 may be configured to control operation of the SEOR component(s) 130 of the sanitization device 110, which may comprise, inter alia, selectively coupling SEOR component(s) 130 to power (e.g., to power supplied by the power module 111 and/or other suitable power source). The control componentry 112 may be further configured to monitor respective SEOR component(s) 130. As illustrated in FIG. 1B the control componentry 112 may comprise and/or be coupled to one or more monitoring and/or control elements, such as a power element 102. The power element 102 may be configured to selectively couple one or more SEOR component(s) 130 to power (e.g., couple a particular SEOR component 130 to power, as illustrated in FIG. 1B). The power element 102 may comprise a coupling element, the coupling element comprising means for selectively coupling an SEOR component 130 to power which may include, but is not limited to: a coupler, coupling circuitry, a bus, bus circuitry, a switch, switch circuitry, an interposer, interposer circuitry, control circuitry, a power controller, power control circuitry, a regulator, regulation circuitry, voltage regulation circuitry, current regulation circuitry, and/or the like. The control componentry 112 may utilize the power element 102 (and/or coupling element thereof) to selectively activate the particular SEOR component 130 (e.g., selectively couple and/or decouple the particular SEOR element 102 from power). Alternatively, or in addition, the power element 102 may be configured to monitor electrical characteristics of the particular SEOR component 130; the power element 102 may comprise a measurement element, the measurement element comprising means for measuring, acquiring, capturing, detecting, sensing, monitoring and/or otherwise determining electrical characteristics of an SEOR component 130, which may include, but are not limited to: sense circuitry, metering circuitry, a power meter, power sense circuitry, a current meter, current sense circuitry, a low side current sense, a high side current sense, a voltage meter, voltage sense circuitry, an impedance meter, impedance sense circuitry, a resistance meter, resistance sense circuitry, and/or the like. The control componentry 112 may be configured to monitor electrical characteristics of the particular SEOR component 130 by use of the power element 102 (and/or measurement element thereof). The control componentry 112 may attempt to activate the particular SEOR component 130 by, inter alia, coupling the particular SEOR component 130 to power, and may determine whether the SEOR component 130 is active based on electrical characteristics of the particular SEOR component 130 determined by use of, inter alia, the power element 102 (e.g., determine whether the particular SEOR component 130 activated and/or is producing sanitizing EO radiation 135). SEOR component(s) 130 that are functional and/or active may exhibit baseline electrical characteristics. The baseline electrical characteristics of an SEOR component 130 may correspond with nominal operation of the SEOR component 130. The baseline electrical characteristics of an SEOR component 130 may be based on one or more of: the type of the SEOR component 130, specifications of the SEOR component 130 (e.g., manufacturer specifications), the state and/or configuration of the SEOR component 130 (e.g., the type of EO radiation being produced by the SEOR component 130, output power level, input power level, wear endured by the SEOR component 130, and/or the like), testing and experience, and/or the like. In some embodiments, the baseline electrical characteristics of an SEOR component 130 may comprise one or more thresholds and/or ranges, such as a current threshold, an impedance threshold, and/or the like. The control componentry 112 may be configured to determine whether the particular SEOR component 130 is functional and/or successfully activated in response to determining that electrical characteristics of the particular SEOR component 130 (as determined by, inter alia, the power element 102) correspond with the baseline characteristics thereof, and may determine that the particular SEOR component 130 is non-functional and/or failed to activate in response to determining that the electrical characteristics deviate from the baseline electrical characteristics. The control componentry 112 may determine whether the particular SEOR component 130 is functional and/or successfully activated in response to comparing a low side current sensed at the particular SEOR component 130 to a low side current threshold and/or range.

In some embodiments, the control componentry 112 may further comprise and/or be communicatively coupled to an EO radiation sensor 103, which may be configured to detect and/or measure sanitizing EO radiation 135 emitted by respective SEOR components 130. The EO radiation sensor 103 may be disposed within the interior compartment 122 of the enclosure 120 (and/or be optically coupled thereto). The control componentry 112 may be configured to determine characteristics of sanitizing EO radiation emitted by the particular SEOR component 130 by use of the EO radiation sensor 103. The control componentry 112 may be configured to implement diagnostic operations comprising selectively activating selected SEOR components 130 and measuring sanitizing EO radiation 135 produced thereby. A diagnostic operation on the particular SEOR component 130 may comprise selectively activating the particular SEOR component 130 while other(s) of the SEOR component(s) 130 are deactivated and measuring sanitizing EO radiation 135 thereby by use of the EO radiation sensor 103. The diagnostic operation may comprise measuring sanitizing EO radiation 135 produced in response to different input power levels, different output power levels, different wavelengths and/or spectrum, and/or the like.

Referring back to FIG. 1A, the control componentry 112 is further configured to determine, monitor, and/or maintain information pertaining to the status of the sanitization device 110 (sanitization device metadata 113). The sanitization device (SD) metadata 113 may be maintained within memory and/or storage resources of the control componentry 112, such as a register, on-board memory, volatile memory, RAM, firmware storage, and/or the like. The SD metadata 113 may comprise any suitable information pertaining to the sanitization device 110, functionality of the sanitization device 110, diagnostics pertaining to components of the sanitization device 110 (e.g., the status of respective SEOR components 130, as disclosed herein), and/or the like. The SD metadata 113 may indicate whether the sanitization device 110 is currently implementing a sanitization operation (e.g., whether one or more SEOR components 130 are currently active), may indicate the status of a current sanitization operation (e.g., track the progress of the current sanitization operation), and/or the like. The SD metadata 113 may further indicate whether the sanitization device 110 (and/or access component 124 thereof) is currently in the open, closed, and/or locked configuration, as disclosed herein. In some embodiments, the SD metadata 113 may be further configured to indicate whether the sanitization device 110 is capable of implementing sanitization operations (e.g., based on the status of respective SEOR component(s) 130). In some embodiments, the SD metadata 113 is further configured to indicate a status of respective SEOR components 130, such as whether one or more SEOR component(s) 130 of the sanitization device 110 are in need of replacement, remaining life of respective SEOR component(s) 130, and/or the like. The control componentry 112 may identify failed and/or non-functional SEOR component(s) 130 in response to, inter alia, monitoring electrical characteristics of the SEOR components 130. A failed, non-functional, or “burnt out” SEOR component 130 may exhibit electrical characteristics substantially similar to an open circuit and/or capacitor (as opposed to a functional SEOR component 130 that conforms with determined baseline characteristics, as disclosed herein).

The control componentry 112 may comprise and/or be operatively and/or communicatively coupled to human-machine interface (HMI) components 114. The HMI components 114 may comprise any suitable means for human-machine interaction including, but not limited to: input/output devices, such as keyboards, buttons, switches, pointer and/or gesture devices (e.g., mouse, touch pad, touch screen, and/or the like), cameras, display devices, monitors, status indicators, static indicator lights, LED lights, audio capture devices, audio output devices, haptic feedback devices, and/or the like. As illustrated in FIG. 1A, one or more of the HMI components 114 may be disposed on the access component 124 of the sanitization device 110. The disclosure is not limited in this regard, however, and could be adapted to place HMI components 114 at any suitable location and/or any suitable configuration.

The HMI components 114 may comprise a status element 115, which may be configured to indicate portions of the SD metadata 113 maintained by the control componentry 112, as disclosed herein. The status element 115 may be configured to indicate whether the sanitizing device 110 is capable of implementing sanitization operations, whether the sanitization device 110 is currently implementing a sanitization operation, a status and/or progress of the current sanitization operation, whether the access component 124 is locked (e.g., whether the access component 124 can be transitioned to the open configuration), and/or the like. The HMI components 114 may further comprise an input element 116. The input element 116 may be configured to receive user commands, which may include, but are not limited to commands to: initiate a sanitization operation, interrupt a sanitization operation, configure the sanitization device 110, and/or the like. Interrupting a sanitization operation may comprise terminating the sanitization operation by, inter alia, deactivating the SEOR components 130, unlocking the access component 124 (and/or closure component 126), and/or the like. Configuring the sanitization device 110 may comprise one or more of: configuring sanitization functionality of the sanitization device 110 (e.g., specifying the duration, intensity, and/or characteristics of sanitizing EO radiation applied during respective sanitization operations), configuring an operating mode sanitization device 110, and/or the like. Configuring the operating mode may comprise specifying one of a manual mode and an automatic mode. In the manual mode, the control componentry 112 may be configured to initiate sanitization operations in response to: a) determining that the sanitization device 110 is in the closed configuration (e.g., the access component 124 is closed and/or locked), and b) receiving a start command through the input element 116 (and/or another HMI component 114). In the automatic mode, the control componentry 112 may be configured to automatically initiate sanitization operations in response to determining that the sanitization device 110 is in the closed configuration (without receiving a start command and/or other user interaction).

Implementing a sanitization operation on a target object 10 may comprise executing a specified sanitization cycle (or sanitization procedure), comprising one or more of: irradiating the target object 10 with sanitizing EO radiation 135 for a determined time period, irradiating the target object 10 with a determined amount and/or cumulative energy of sanitizing EO radiation 135, irradiating the target object 10 with sanitizing EO radiation 135 having specified characteristics (e.g., specified wavelength, band, type, intensity, and/or power level), and/or the like. Implementing a sanitization operation may, therefore, comprise selectively activating SEOR component(s) 130 of the sanitization device 110. Implementing a sanitization operation may further comprise: verifying that the access component 124 is in the closed configuration and implementing a sanitization cycle in response to the verifying, as disclosed herein. In some embodiments, the control componentry 112 is further configured to lock the sanitization device 110 (e.g., lock the access component 124 and/or closure component 126) during sanitization operations. The control componentry 112 may be further configured to monitor the state of the access component 124 during sanitization operations and may interrupt sanitization operations in response to detecting that the access component 124 is not in the closed and/or locked configuration. Alternatively, or in addition, the control componentry 112 may be configured to interrupt a sanitization operation in response to an interrupt command received through the HMI components 114 (e.g., the input element 116).

In some embodiments, the control componentry 112 may further comprise and/or be coupled to a communication interface 118 configured to, inter alia, couple the sanitization device 110 to a network 101. The network 101 may comprise any suitable means for electronic communication, including, but not limited to: an Internet Protocol (IP) network, the Internet, a wireless network, a Local Area Network (LAN), a Wide Area Network (WAN), a Virtual Private Network (VPN), a wireless network (e.g., IEEE 802.11a-n wireless network, Bluetooth® network, Near-Field Communication (NFC) network, and/or the like), a public switched telephone network (PSTN), a mobile network (e.g., a network configured to implement one or more technical standards or communication methods for mobile data communication, such as Global System for Mobile Communication (GSM), Code Division Multi Access (CDMA), CDMA2000 (Code Division Multi Access 2000), EV-DO (Enhanced Voice-Data Optimized or Enhanced Voice-Data Only), Wideband CDMA (WCDMA), High Speed Downlink Packet Access (HSDPA), High Speed Uplink Packet Access (HSUPA), Long Term Evolution (LTE), Long Term Evolution-Advanced (LTE-A), and/or the like), a combination of networks, and/or the like. In some embodiments, the control componentry 112 may be configured to communicate status and/or configuration information pertaining to the sanitization device 110 via the network 101. The control componentry 112 may be further configured to receive commands and/or configuration information through the network 101 (e.g., may be configured through the network, receive commands to implement sanitization operations through the network 101, and/or the like).

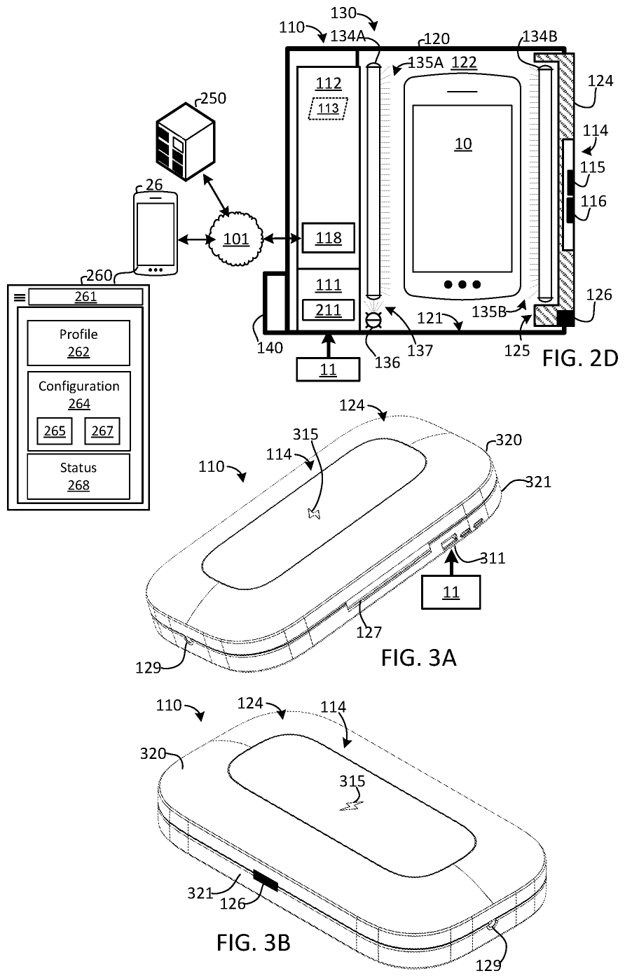

FIG. 2A is a schematic block diagram of further embodiments of a sanitization device 110, as disclosed herein (e.g., second embodiments). In the FIG. 2A embodiment, the sanitization device 110 comprises a CCFL 134 (the SEOR component(s) 130 of the sanitization device 110 comprise a CCFL 134). Although FIG. 2A depicts the SEOR component(s) 130 comprising a single CCFL 134, the disclosure is not limited in this regard and could be adapted to include any number of SEOR components 130 of any suitable type disposed at any suitable location within the sanitization device 110. The control componentry 112 may be adapted to configure the CCFL 134 to selectively emit sanitizing EO radiation 135 within the interior compartment 122 of the enclosure 120, as disclosed herein. The control componentry 112 may be further configured to selectively activate respective CCFL 134, monitor respective CCFL 134 and/or maintain SD metadata 113 pertaining to the status of the sanitization device 110 and/or SEOR component(s) 130 thereof (e.g., CCFL 134), as disclosed herein.

FIG. 2B is a schematic block diagram illustrating further embodiments of the disclosed sanitization device 110. As illustrated, the control componentry 112 may comprise and/or be coupled to one or more monitoring and/or control elements, such as a power element 102. The power element 102 may be configured to selectively couple a CCFL 134 to power and/or monitor electrical characteristics of the CCFL 134, as disclosed herein. The control componentry 112 may be configured to determine whether the CCFL 134 is in an active state by, inter alia, evaluating electrical characteristics of the CCFL 134 acquired by the power element 102 (e.g., may comprise comparing the acquired electrical characteristics to baseline characteristics of the CCFL 134, such as a low side current threshold, and/or the like). Alternatively, or in addition, the control componentry 112 may be configured to measure sanitizing EO radiation 135 produced by the CCFL 134 by use of, inter alia, an EO radiation sensor 103 (not shown in FIG. 2B to avoid obscuring details of the illustrated embodiments).

As illustrated in FIG. 2B, the CCFL 134 may comprise and/or be coupled to supply circuitry 234, which may be configured to produce power suitable for the CCFL 134 (condition, regulate, transform, process and/or otherwise produce power having suitable characteristics). The supply circuitry 234 may comprise power conversion circuitry, a transformer, and/or the like. The CCFL 134 and corresponding supply circuitry 234 may be disposed within the sanitization device 110 (e.g., within the enclosure 120 and/or one or more other compartments). Components of the sanitization device 110 may be damaged by high temperature conditions. By way of non-limiting example, high temperature conditions may damage materials comprising the sanitization device 110 and/or enclosure 120 (e.g., may melt plastic elements of the sanitization device 110 and/or enclosure 120), reduce the reflectivity of the inner surface 121 of the enclosure 120 (e.g., damage and/or deform the inner surface 121 and/or reflective coating thereof), damage control componentry 112 of the sanitization device 110 (e.g., damage circuitry, HMI components 114, and/or the like), damage the SEOR component(s) 130 of the sanitization device 110 (e.g., damage the CCFL 134 and/or supply circuitry 234 thereof), and/or the like. Some types of SEOR component(s) 130 may produce a significant amount of heat during operation. The sanitization device 110 may be compact and/or enclosed and, as such, may not be capable of dissipating heat produced by the SEOR component(s) 130 at a rate sufficient to avoid high temperature conditions, particularly when the sanitization device 110 is used continuously, under high ambient temperature conditions, in areas with limited air circulation, and/or the like.

In some embodiments, the control componentry 112 is configured to prevent heat-related damage to the sanitization device 110. The control componentry 112 may comprise and/or be coupled to a temperature sensor 104, which may be configured to monitor a temperature of respective SEOR component(s) 130, such as the CCFL 134 (and/or supply circuitry 234 thereof). The temperature sensor 104 may comprise any suitable means for sensing, measuring, acquiring, and/or otherwise monitoring temperature including, but not limited to: a thermistor, a Negative Temperature Coefficient (NTC) thermistor, and/or the like. In some embodiments, the temperature sensor 104 may be configured to measure a temperature of a transformer of the supply circuitry 234 (e.g., may be coupled and/or attached to the supply circuitry and/or transformer thereof). In some embodiments, the control componentry 112 is configured to deactivate SEOR component(s) 130 that exceed and/or satisfy one or more high temperature thresholds (the control componentry 112 may be configured to deactivate the CCFL 134 in response to determining that the temperature of the CCFL 134 and/or supply circuitry 234 exceeds one or more high temperature thresholds). The high temperature threshold(s) may be set in accordance with one or more of: characteristics of the SEOR component 130 (e.g., an operating temperature range specified by a manufacturer of the SEOR component 130), heat-related characteristics of components of the sanitization device 110 (e.g., temperature at which the component is susceptible to heat-related damage, such as a melt and/or deformation temperature of materials comprising the enclosure 120, inner surface 121, and/or the like), testing and experience, and/or the like. Alternatively, or in addition, the control componentry 112 may be configured to monitor temperatures of respective SEOR components 130 (and/or temperatures at designated location(s) within the sanitization device 110) and/or implement temperature mitigation operations in response to the monitoring. The temperature mitigation operations may comprise any operation capable of, inter alia, reducing the temperature within the sanitization device 110 (and/or respective SEOR component(s) 130 thereof) including, but not limited to: reducing a power output of one or more SEOR component(s) 130, deactivating one or more SEOR component(s) 130, interrupting a sanitization operation being implemented by the sanitization device 110, modifying the sanitization operation, pausing the sanitization operation, and/or the like. Modifying a sanitization operation may comprise changing the intensity and/or duration of sanitizing EO radiation used therein. The modifying may comprise reducing an intensity of the sanitizing EO radiation by, inter alia, reducing a power output of one or more SEOR component(s) 130 (and/or deactivating one or more of the SEOR component(s) 130). The modifying may further comprise increasing a duration of the sanitization operation such that a cumulative energy of sanitizing EO radiation 135 generated during the sanitization operation is substantially unchanged due to the modifications. Pausing a sanitization operation may comprise interrupting the sanitization operation and resuming the sanitization operation after a determined time period (and/or in response to the monitored temperature falling below a temperature threshold).

Referring back to FIG. 2A, some types of SEOR components 130 may be susceptible to dormancy conditions (e.g., may be susceptible to becoming dormant and/or transitioning to a dormant state). As used herein, a “dormancy condition” or “dormant state” of an SEOR component 130 refers to a state in which the SEOR component 130 is incapable of being activated through normal means (e.g., by coupling the SEOR component 130 to power, as disclosed herein). When coupled to power, a dormant SEOR component 130 may remain in an inactive state (e.g., fail to generate sanitizing EO radiation 135). The electrical characteristics of a dormant SEOR component 130 may deviate from baseline characteristics of functional, non-dormant SEOR components 130. The electrical characteristics of a dormant SEOR component 130 may be substantially similar to the electrical characteristics of a failed and/or burnt out SEOR component 130 (e.g., may be substantially similar to an open circuit and/or capacitor). A dormant SEOR component 130 may, therefore, appear to have failed or burnt out (based on the electrical characteristics thereof, as disclosed herein).

Certain types of SEOR component 130 may become dormant due to, inter alia, being isolated from suitable external stimulus for a sufficient period of time. As used herein, “external stimulus” or “suitable external stimulus” refers to a specified type of EO radiation capable of preventing a particular type of SEOR component 130 from becoming dormant (e.g., may refer to particular EO radiation wavelength(s) and/or spectrum). A dormant SEOR component 130 may be returned to a functional, non-dormant state by, inter alia, exposing the dormant SEOR component 130 to suitable external stimulus. The SEOR component 130 may be prevented from becoming dormant by, inter alia, periodic exposure to suitable external stimulus. Different types of SEOR component(s) 130 may require different types of external stimulus. The sanitizing EO radiation 135 emitted by the SEOR component(s) 130 of the sanitization device 110 may not be capable of preventing certain types of SEOR component(s) 130 from becoming dormant and/or recovering from dormancy (e.g., may lack certain EO radiation wavelength(s) and/or the like). By way of non-limiting example, the CCFL 134 may require external stimulus in the visible spectrum to avoid dormancy, which may not be included in the sanitizing EO radiation 135 produced by the SEOR component(s) 130 of the sanitization device 110 and/or the CCFL 134 itself (and/or may not be produced at sufficient intensity and/or in sufficient amounts). When a CCFL 134 is not exposed to sufficient suitable external stimulus, the CCFL 134 may transition to a low electron state that can cause the CCFL 134 to fail to fire and/or activate when the CCFL 134 is coupled to power (a dormant state). The SEOR component(s) 130 of the sanitization device 110 may be enclosed within the interior compartment 122 for extended periods of time, during which the SEOR component(s) 130 may be substantially isolated from suitable external stimulus. SEOR component(s) 130 that are subject to dormancy, such as CCFL 134, may become dormant over time, particularly if the sanitation device 110 utilized and/or opened infrequently. As disclosed above, a dormant SEOR component 130 may exhibit electrical characteristics that are substantially similar to those of a failed SEOR component 130 (e.g., substantially similar to an open circuit and/or capacitor). A dormant SEOR component 130 may, therefore, be misidentified as a failed component, which may result in unnecessary maintenance (and/or replacement of dormant, but otherwise potentially functional SEOR components 130).

In the FIG. 2A embodiment, the control componentry 112 may be configured to prevent SEOR components 130 from becoming dormant (and/or recover dormant SEOR components 130), thereby preventing unnecessary maintenance and/or replacement. The control componentry 112 may prevent and/or recover from dormancy conditions by, inter alia, exposing the SEOR components 130 to suitable external stimulus. As illustrated in FIG. 2A, the sanitization device 110 may comprise an external stimulus (ES) component 136, which may comprise any suitable means for producing EO radiation, as disclosed herein (e.g., an LED or the like). The ES component 136 may be configured to produce recovery EO radiation 137, which may be configured to prevent SEOR component(s) 130 of the sanitization device 110 from becoming dormant (and/or recover from dormancy). In the FIG. 2A embodiment, the ES component 136 is configured to emit recovery EO radiation 137 comprising suitable external stimulus for one or more of the SEOR components 130, such as the CCFL 134. The ES component 136 may be configured to generate recovery EO radiation 137 capable of preventing the CCFL 134 from becoming dormant (and/or transition the CCFL 134 from a dormant state to a functional, non-dormant state). The recovery EO radiation 137 may comprise wavelength(s) not included in the sanitizing EO radiation 135 produced by the SEOR components 130 (e.g., may comprise EO radiation in the visible spectrum). The ES component 136 may be further configured to produce recovery EO radiation 137 comprising suitable external stimulus for other types of SEOR component(s) 130, which may differ from the external stimulus required by the CCFL 134. Alternatively, or in addition, the sanitization device 110 may comprise a plurality of ES components 136, each configured to provide recovery EO radiation 137 suitable for respective type(s) of SEOR component(s) 130.

In some embodiments, the control componentry 112 is configured to activate the ES component 136 in response to activating one or more SEOR component(s) 130 (e.g., in response to attempting to activate the CCFL 134). The ES component 136 may be activated substantially concurrently with activation of the CCFL 134. Alternatively, the control componentry 112 may be configured to activate the ES component 136 prior to activation of the CCFL 134 (may “prime” the CCFL 134 prior to activation). In some embodiments, the control componentry 112 is configured to deactivate the ES component 136 after a determined time period and/or in response to activation of the CCFL 134. Alternatively, the ES component 136 may remain active while the CCFL 134 is active.

FIG. 2C is a schematic block diagram of another embodiment of a sanitization device 110, as disclosed herein. In the FIG. 2C embodiment, the control componentry 112 comprises an EO component controller (controller 212) configured to, inter alia, manage, regulate, monitor, and/or otherwise control SEOR component(s) 130 of the sanitization device 110, as disclosed herein. The controller 212 may be configured to interface with, selectively activate, and/or monitor the SEOR component(s) 130 and/or ES component 136, as disclosed herein (e.g., by use of respective power element(s) 102 coupled thereto, as disclosed herein). The controller 212 may be further configured to detect activation failures of respective SEOR component(s) 130 based on, inter alia, electrical characteristics of the SEOR component(s) 130 determined by use of, inter alia, measurement element(s) coupled to the respective SEOR component(s) 130, as disclosed herein. The controller 212 may detect an activation failure of an SEOR component 130 in response to determining that current flow through the SEOR component 130 is lower than a threshold while the SEOR component 130 is coupled to power (and/or impedance of the SEOR component 130 exceeds a threshold).

The controller 212 may comprise a recovery module 214 configured to, inter alia, prevent SEOR component(s) 130 of the sanitization device 110 from becoming dormant and/or recover from activation failures due to dormancy. The recovery module 214 may be configured to cause the controller 212 to activate the ES component 136 concurrently with (and/or prior to) activation of one or more SEOR component(s) 130, as disclosed herein. The recovery module 214 may be further configured to cause the controller 212 to deactivate the ES component 136 after a determined time period and/or in response to the controller 212 determining that the SEOR component(s) 130 were successfully activated (e.g., based on electrical characteristics of the SEOR component(s) 130, as disclosed herein). Alternatively, the recovery module 214 may configure the controller 212 to maintain the ES component 136 in the active state while the SEOC component(s) 130 remain active.

In some embodiments, the recovery module 214 is configured to selectively activate the ES component 136 (by use of the controller 212). The recovery module 214 may activate the ES component 136 in response to the controller 212 detecting an activation failure pertaining to a particular SEOR component 130. The recovery module 214 may configure the controller 212 to maintain the ES component 136 in the active state for a determined time period (a recovery period), until the particular SEOR component 130 activates, and/or while the particular SEOR component 130 is coupled to power. The recovery module 214 may be further configured to distinguish dormant SEOR components 130 from failed SEOR components 130. The recovery module 214 may determine that the activation failure was due to dormancy in response to the particular SEOR component 130 activating in response to the recovery EO radiation 137. The recovery module 214 may determine that the particular SEOR component 130 has failed (and/or requires maintenance or replacement) in response to the particular SEOR component 130 failing to activate in response to the recovery EO radiation 137. The recovery period may be set in accordance with characteristics of the particular SEOR component 130 and/or recovery EO radiation 137 produced by the ES component 136 (e.g., based on an estimate of the time required for the particular SEOR component 130 to recover from dormancy in response to the recovery EO radiation 137). Alternatively, or in addition, the recovery period for respective types of SEOR component(s) 130 may be determined and/or adjusted in accordance with testing and experience.

In some embodiments, the recovery module 214 may be configured to cause the controller 212 to periodically activate the ES component 136 (and/or configure the ES component 136 to produce recovery EO radiation 137 comprising suitable external stimulus for the SEOR component(s) 130). The periodic activation of the ES component 136 may prevent the SEOR component(s) 130 from becoming dormant due to long periods of isolation from suitable external stimulus, as disclosed herein. The periodic activation of the ES component 136 may prevent delays due to recovery when the sanitization device 110 is subsequently used to implement a sanitization operation. The recovery module 214 may be configured to activate the ES component 136 at any suitable period and/or interval (e.g., daily, weekly, monthly, and/or the like). In some embodiments, the recovery module 214 is configured to determine whether to periodically activate the ES component 136 based on, inter alia, the availability of power resources. The recovery module 214 may periodically activate the ES component 136 while the sanitization device 110 is coupled to an external power source 11 and may delay (and/or defer) period activation otherwise.

In FIG. 2C, the power module 111 may comprise an internal power source 211, as disclosed herein (e.g., power storage means, such as battery or the like). The recovery module 214 may be configured to cause the controller 212 to periodically activate the ES component 136 (and/or configure the ES component to produce recovery EO radiation 137 comprising suitable external stimulus for the SEOR component(s) 130 of the sanitization device 110) while disconnected from the external power (when disconnected from the external power source 11). The recovery module 214 may periodically activate the ES component 136 by use of power supplied by the internal power source 211. The recovery module 214 may increase the time duration between activations (and/or decrease the duration for which the ES component 136 is activated) while relying on the interval power source 211. The ES component 136 may be configured to further reduce the power requirements of the periodic activations based on, inter alia, an amount of power available in the internal power source 211 and may cease the periodic activations in response to the amount of available power falling below a threshold.

FIG. 2D is a schematic block diagram of further embodiments of the disclosed sanitization device 110 (further embodiments of the second embodiments of the sanitization device 110). In the FIG. 2D embodiment, the SEOR components 130 of the sanitization device 110 comprise a first CCFL 134A configured to produce first sanitizing EO radiation 135A and a second CCFL 134B configured to produce second sanitizing EO radiation 135B. The first sanitizing EO radiation 135A and the second sanitizing EO radiation 1356 may comprise a same type of EO radiation (same or similar wavelength(s) and/or spectrum). Alternatively, the first sanitizing EO radiation 135A produced by the first CCFL 134A may differ from the second sanitizing EO radiation 135B produced by the second CCFL 1346. The second CCFL 134B may be disclosed on and/or within the access component 124 of the enclosure 120. The disclosure is not limited in this regard and could be adapted to include SEOR component(s) 130 at any suitable location and/or of any suitable type. The control componentry 112 may comprise a controller 212 configured to configure, activate, and/or monitor the first CCFL 134A and/or second CCFL 134B, as disclosed herein. The controller 212 may comprise a recovery module 214 configured to prevent and/or recover from activation failures of one or more of the first CCFL 134A and/or second CCFL 134B. The recovery module 214 may be configured to prevent activation failures by, inter alia, configuring the ES component 136 to emit recovery EO radiation 137 in response to attempting to activate one or more of the first CCFL 134A and/or the second CCFL 134B. The recovery module 214 may be configured to adapt the recovery EO radiation 137 in accordance with characteristics of the first CCFL 134A and/or the second CCFL 134B. In some embodiments, the first CCFL 134A requires first external stimulus and the second CCFL 134B requires second external stimulus, different from the first external stimulus. The recovery module 214 may be adapted to configure the ES component 136 to produce recovery EO radiation 137 comprising the first external stimulus in response to attempting to activate the first CCFL 134A (and/or in response to an activation failure of the first CCFL 134A) and may configure the ES component 136 to produce recovery EO radiation 137 comprising the second external stimulus in response to attempting to activate the second CCFL 134B (and/or in response to an activation failure of the first CCFL 134B). In some embodiments, the recovery module 214 is adapted to configure the ES component 136 to produce recovery EO radiation 137 comprising both the first external stimulus and the second external stimulus and/or alternative therebetween in response to activation of both the first and second CCFL 134A and 134B (and/or activation failure of both CCFL 134A and 134B). The recovery module 214 may be further configured to cause the ES component to periodically emit recovery EO radiation 137 during inactivity, as disclosed herein (e.g., may configure the ES component 136 to periodically produce one or more of the first external stimulus and/or second external stimulus).

In some embodiments, the sanitization device 110 may further comprise a security component 140, which may be configured to secure the sanitization device 110 at a particular location and/or securely attach the sanitization device 110 to a particular object (e.g., a kiosk, desk, stand, and/or the like). The security component 140 may comprise any suitable locking means, as disclosed herein (e.g., a mechanical lock, a cable lock, an electronic lock, a magnetic lock, a network-accessible locking mechanism, and/or the like). The security component 140 may be further configured to detect compromise events, such as attempts to tamper with, bypass, and/or otherwise disable security functionality thereof. The security component 140 may be communicatively and/or operatively coupled to the control componentry 112, which may monitor a status of the security component 140 (e.g., whether the security component 140 is locked, released, and/or the like), detected compromise events, and/or the like. Information pertaining to the security component 140 may be maintained with the SD metadata 113, as disclosed herein.

The control circuit may be configured to provide status information, attributes, and/or sensor information to a management system (e.g., management system 250). The control circuit may communicate via a primary network to the management system (e.g., the Internet via Wi-Fi, cellular, or other network access technology) or use one or more relays to communicate with the management system (e.g., connecting with a first network (e.g., personal area network like Bluetooth®, etc.) to a device that is capable of transmitting via a second network (e.g., Wi-Fi, cellular, etc.)). Messages may comprise information, instructions, and/or attributes (e.g., location, battery status, lock status, account information, owner information, etc.). In some embodiments, the control circuit may identify the device and/or attributes of the device within the sanitation system and report the detected device to the management system. The device identity may be a MAC Address, device type, device brand, device model, cellular identifier, weight, dimensions, color, or other identifying information.

For example, a security sensor may detect a disconnection of a power source from a sanitation device. The control circuitry may receive an indicator of the detection from the security sensor. The control circuitry may send, via Bluetooth®, a message indicating the removal of power and/or other attributes (e.g., location, battery status, lock status, account information, owner information, etc.) to a paired cellular phone. The cellular phone may receive the message and provide the message to an application executing on the cellular phone. The application may process the message and cause the cellular phone to send, via a cellular radio access technology, a message (e.g., relay the message) comprising the indication to a management system. The cellular phone may be inside the sanitation device or within reception range of the sanitation device.

Similarly, a management system may send messages to the sanitation device, either directly or through a relay. The management system messages may comprise requests for information, commands, configurations, or other information and/or instructions.

As disclosed above, the control componentry 112 may comprise and/or be coupled to a network interface 118. The network interface 118 may be configured to couple the control componentry 112 to a network 101, as disclosed herein. In some embodiments, the control componentry 112 may be configured to report status information pertaining to the sanitization device 110 to a network-accessible management service (management system 250). The control componentry 112 may be configured to report any suitable status information, as disclosed herein (e.g., the control componentry 112 may transmit the SD metadata 113 to the management system 250 and/or portions thereof). In some embodiments, the status information may comprise a location and/or security status of the sanitization device 110. The control componentry 112 may be configured to determine and/or monitor a location of the sanitization device 110 by use of the network interface 118 (e.g., by use of a GPS network). The control componentry 112 may be configured to determine and/or monitor the security status of the sanitization device 110 by use of the security component 140, as disclosed above. The control componentry 112 may be configured to alert the management system 250 in response to compromise events detected by the security component 140. The control componentry 112 may be further configured to alert the management system 250 of other types of compromise events, such as detecting that the sanitization device 110 is being moved outside of specified bounds (outside of a specified geofence), the sanitization device 110 has been disconnected from the external power source 11, the security component 140 has been compromised or destroyed, and/or the like. The alerting may further comprise producing alerts at the sanitization device 110 itself (through one or more HMI components 114), such as audible alerts, visual alerts, haptic alerts, and/or the like. The control componentry 112 may be configured to report status information and/or issue alerts of compromise events in the absence of external power (e.g., while disconnected from the external power source 11). In response to a compromise event, the control componentry 112 may be configured to periodically (or continuously) attempt to report the location and/or status of the sanitization device 110 to the management system 250 by use of power supplied by the internal power source 211. The control componentry 112 may continue such reporting until the internal power source 211 is exhausted.

In some embodiments, the control componentry 112 may be further configured to interface with computing device(s) through the network 101. The control componentry 112 may be configured to interface with an application 260 operating on a mobile computing device 16 through, inter alia, Bluetooth, NFC, WiFi, and/or the like. The application 260 may be hosted by the sanitization device 110 (may comprise a hosted application, such as a web-based application). Alternatively, or in addition, the application 260 may comprise a native and/or installed application configured for operation on the mobile computing device 16. The application 260 may comprise a visual identifier 261 of the sanitization device 110 (a name, location, brand, and/or the like). The application may further comprise a profile component 262 configured to enable users to register with the sanitization device 110 (and/or management system 250). A profile component 262 may enable a user to register information pertaining to device(s) of the user, information pertaining to sanitization operations performed on the device(s) by the sanitization device 110 (and/or other sanitization devices 110 associated with the management system 250), and/or the like. The profile component 262 may be further configured to enable users to pay for access to the sanitization device 110.

A configuration component 264 may provide for configuring operation of the sanitization device 110. The configuration component 264 may provide for specifying a sanitization operation to perform on a target object 10, configure the sanitization device 110 to unlock the access component 124, initiate a sanitization operation, interrupt and/or terminate a sanitization operation, and/or the like. The configuration component 264 may comprise a start component 265, which may be configured to enable users to initiate a sanitization operation using the sanitization device 110. The start component 265 may enable a user to unlock the sanitization device 110 (unlock the access component 124 and/or closure component 126) and/or initiate a sanitization operation in response to the user closing the sanitization device 110 (and/or issuing a start command through the HMI component(s) 114 and/or start component 265). In some embodiments, the configuration component 264 may further comprise a security component 265 configured to enable users to specify security information pertaining to user interaction with the sanitization device 110. The security component 265 may enable a user to establish a user-specific unlock code. The control componentry 112 may be configured to use the unlock code of the user to secure the sanitization device 110 during sanitization operations initiated by the user. Implementing a sanitization operation for a particular user may comprise: the particular user interacting with the configuration component 264 to configure and/or initiate the sanitization operation, which may comprise establishing an unlock code for the particular user by use of the security component 267 and/or unlocking the sanitization device 110 for use by the particular user by use of the start component 265; the sanitization device 110 implementing the specified sanitization operation in response to the user placing the target object 10 within the enclosure 120 and/or locking the sanitization device 110; and maintaining the sanitization device 110 in the locked configuration until completing the sanitization operation and/or receiving the unlock code of the particular user.

In some embodiments, the application 260 further comprises a status component 268, which may be configured to provide status information pertaining to the sanitization device 110, as disclosed herein. The status component 268 may be configured to indicate whether the sanitization device 110 is available for use by the user (e.g., is functional and/or not currently implementing a sanitization operation). The status component 268 may be further configured to indicate a status of sanitization operation(s) currently being implemented by the sanitization device 110. The status component 268 may comprise a progress indicator and/or display time remaining until completion of the current sanitization operations. In some embodiments, the status component 268 may be further configured to enable users to reserve the sanitization device 110. A user may submit a reservation request, which may reserve the sanitization device 110 for use by the user in response to completing the current sanitization operation.

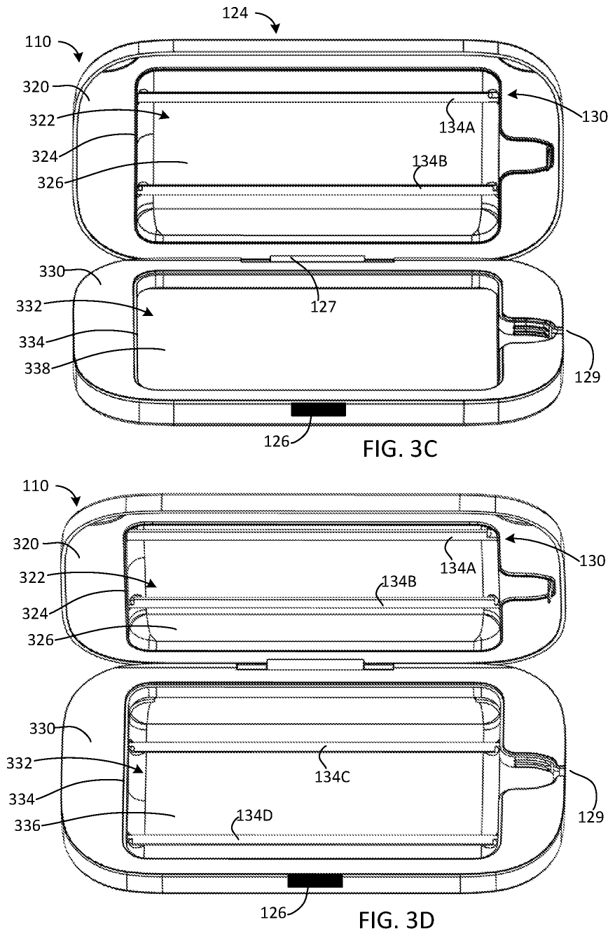

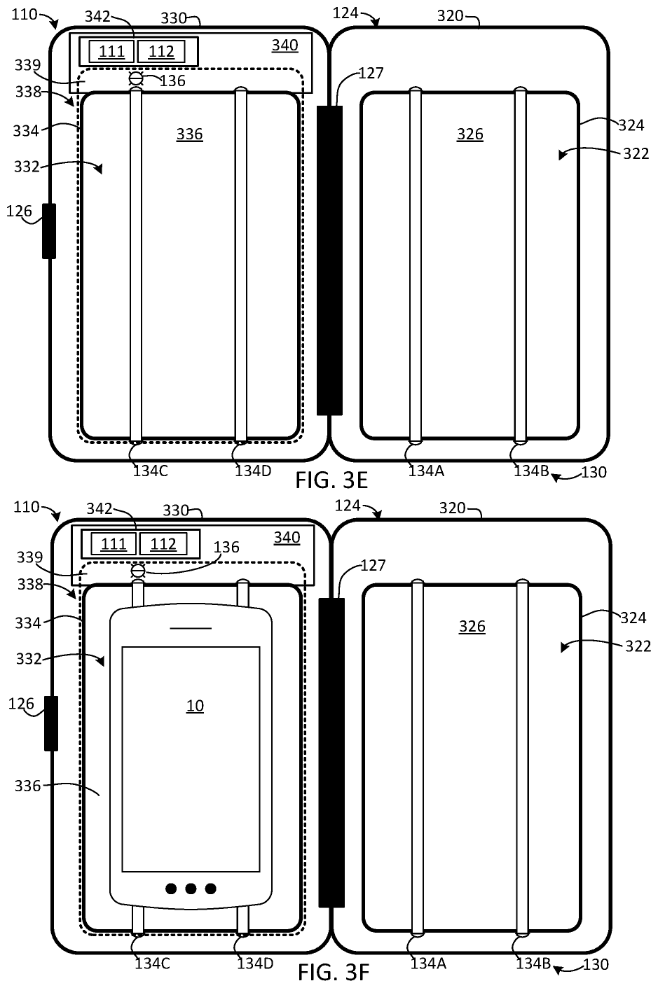

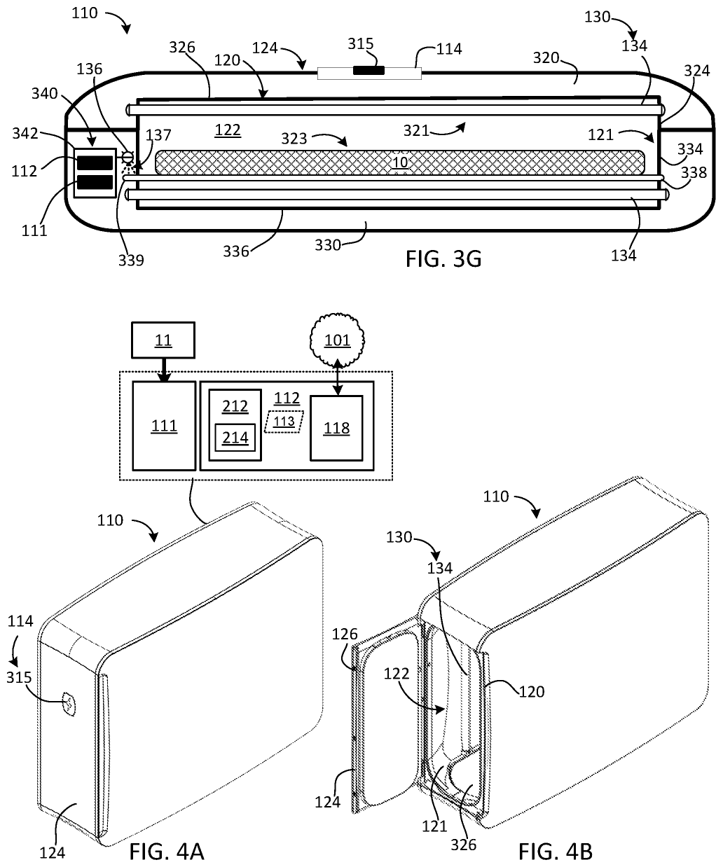

FIGS. 3A and 3B are top front perspective views of further embodiments of a sanitization device 110, as disclosed herein (third embodiments). As illustrated, the sanitization device 110 may comprise a top section 320 coupled to a bottom section 330 in a clamshell configuration. The top section 320 may be coupled to the bottom section 330 by a connection member 127, which may comprise any suitable connection means such as a hinge, flap, joint, and/or the like. As illustrated in further detail herein, the top section 320 and the bottom section 330 may comprise respective interior regions 322 and 332, which may form an interior compartment 122 capable of enclosing a target object 10 for sanitization when in the closed configuration; the top section 320 may, therefore, comprise an access component 124 configured to control physical access to the interior compartment 122 (interior compartment 12 not shown in FIGS. 3A and 3B to avoid obscuring details of the illustrated embodiments). The sanitization device 110 may further comprise a power connector 311 configured to, inter alia, establish an electrical coupling to an external power source 11, as disclosed herein. The sanitization device 110 may further comprise an access port 129 configured to, inter alia, transmit acoustics and to enable a cable to pass into the interior compartment 122. The access port 129 may be configured to prevent transmission of sanitizing EO radiation 135 (e.g., may comprise an absorptive material, a flap, and/or other means for blocking transmission of EO radiation.

The sanitization device 110 may comprise one or more HMI components 114, including an HMI element 315. The HMI element 315 may be configured to indicate a current status of the sanitization device 110, as disclosed herein (e.g., may comprise an LED capable of displaying a plurality of different colors and/or patterns, each corresponding to respective state information). The HMI element 315 may be further configured to receive user input (e.g., may comprise a button, touch pad, and/or the like). The HMI element 315 may be configured to receive user input to set the operating mode of the sanitization device 110 (e.g., automatic, manual, or the like), initiate sanitization operations, configure sanitization operations, and/or the like.

As illustrated in FIG. 3B, the sanitization device 110 may comprise a closure component 126 configured to, inter alia, determine a state of the access component 124 (e.g., whether the access component 124 is open, closed, locked, and/or the like). The closure component 126 may be further configured to selectively secure and/or lock the access component 124 in the closed configuration, as disclosed herein.

FIGS. 3C and 3D are top perspective views of third embodiments of the disclosed sanitization device 110. FIGS. 3C and 3D show the sanitization device 110 in an open configuration. As illustrated the top section 320 may comprise a first interior region 322. The first interior region 322 may comprise a recess defined within the top section 320 by first sidewalls 324 and a first end portion 326. Surfaces of the first sidewalls 324 and first end portion 326 may be configured to reflect and/or transmit sanitizing EO radiation 135, as disclosed herein. The bottom section 330 may comprise a second interior region 332. As illustrated in FIG. 3D, the second interior region 322 may comprise a recess defined within the bottom section 320 by second sidewalls 334 and a second end portion 336 (the second end portion 336 not shown in FIG. 3C to avoid obscuring details of the illustrated embodiments). Surfaces of the second sidewalls 334 and second end portion 336 may be configured to reflect and/or transmit sanitizing EO radiation 135, as disclosed herein.

The sanitization device 110 may comprise a plurality of SEOR components 130, including CCFL 134A and 134B disposed within the first interior region 322 and CCFL 134C and 134D disposed within the second interior region 332 (CCFL 134C-D not shown in FIG. 3C to avoid obscuring details of the illustrated embodiments). As illustrated in FIG. 3C, the bottom section 330 may further comprise a support member 338, which may be configured to receive, hold, and/or secure a target object 10 within the second interior region 332. The support member 338 may be disposed at a determined vertical offset along the second sidewalls 334 (e.g., at a vertical location between the second end portion 336 and ends of the second sidewalls 334). The support member 338 may be disposed above CCFL 134C-D and below the top of the second sidewalls 334. The support member 338 may be configured to transmit sanitizing EO radiation 135. The support member 338 may be comprised of transparent material(s). As used herein, a “transparent material” refers to a material configured to transmit and/or be substantially transparent with respect to sanitizing EO radiation 135 including, but not limited to: glass, plastic, polymer, ceramic, quartz, or other suitable transparent material. The support member 338 may, therefore, be configured to enable sanitizing EO radiation 135 produced by CCFL 134C-D disposed below the support member 338 to pass therethrough. Accordingly, when in the closed configuration, sanitizing EO radiation 135 emitted by the SEOR component(s) 130 may be configured to irradiate substantially all of the first interior region 322 and second interior region 332 due to, inter alia, the position of the CCFL 134A-D, reflectivity of the interior surfaces of the sanitization device 110 (e.g., reflectivity of the first sidewalls 324, first end portion 326, second sidewalls 334, and/or second end portion 336), and transparency of the support member 338.

FIGS. 3E and 3F are top plan views of further embodiments of the disclosed sanitization device 110. In the illustrated embodiments, the bottom section 330 of the sanitization device 110 may further comprise a second compartment or a peripheral compartment 340. As further illustrated in the cross-sectional view of FIG. 3G, the peripheral compartment 340 may be enclosed within the bottom section 330 of the sanitization device 110. The peripheral compartment 340 may comprise componentry of the sanitization device 110, such as the power module 111, control componentry 112, ES component 136, and/or the like. The peripheral compartment 340 may further comprise one or more power elements 102, coupling elements, measurement elements, supply circuitry 234, an internal power source 211, a controller 212, a recovery module 214, communication interface 118, and/or the like (not shown in FIGS. 3E-3G to avoid obscuring details of the illustrated embodiments). The componentry, including the power module 111 and/or control componentry 112, may be disposed on and/or within a peripheral support member 342, such as a package, PCB, and/or the like.

The support member 338 may be disposed within the second interior region 332. The support member 338 may be attached to and/or secured within the second sidewalls 334 by grooves, openings, indentations, ridges, voids, and/or other means. Portions of the support member 338 may extend into and/or through the second sidewalls 334. As illustrated, an end portion 339 of the support member 338 may be configured to protrude through the second sidewalls 334 into the peripheral compartment 340. The end portion 339 may be optically coupled to the ES component 136. The end portion 339 may be configured to transmit recovery EO radiation 137 produced by the ES component 136 from the peripheral compartment 340 into the second interior region 332. The support member 338 thereby can function as a light pipe to transmit recovery EO radiation from the ES component 136 in the peripheral compartment 340 into the primary interior compartment 122. Transmitting recovery EO radiation 137 through the support member 338 may obviate the need for disposing the ES component 136 within the first or second interior regions 322 or 332 and/or forming additional openings within the sidewalls 324/334 or end portions 326/336, which may disrupt reflectivity of the inner surfaces thereof. Utilizing the support member 338 to transmit the recovery EO radiation 137 may, therefore, result in improved distribution and/or coverage sanitizing EO radiation 135 over the target object 10.

FIG. 3F illustrates an embodiment in which a target object 10 is disposed within the second interior region 332. As illustrated, the target object 10 may be secured within the second interior region 332 by the second sidewalls 334 and/or support member 338. The support member 338 may be configured to position the target object above the second end portion 336 of the second interior region 332 (e.g., above the CCFL 134C and 134D).

FIG. 3G is a cross-sectional view of the disclosed sanitization device 110 in the closed configuration. Transitioning to the closed configuration may comprise rotating the top and/or bottom sections 320/330 on the connection member 127 such that the top section 320 couples with the bottom section 330. In the closed configuration, the first interior region 322 and the second interior region 332 may form an interior compartment 122 of the enclosure 120. The interior compartment 122 may be defined by the first and second sidewalls 324/334 and the first and second end portions 326/336 of the top and bottom sections 320/330. The first and second sidewalls 324/334, support member 338, and first end portion 326 may be configured to secure the target object 10 within the interior compartment 122. The support member 338 may be configured to support the target object 10 above SEOR components 130 disposed within the bottom section 330 (e.g., CCFL 134C and 134D, as illustrated in FIGS. 3E and 3F). In some embodiments, the sanitization device 110 may further comprise a top plate configured to, inter alia, prevent the target object 10 from impacting SEOR components 130 disposed within the top section 320 (not shown in FIG. 3G to avoid obscuring details of the illustrated embodiments). The top plate may be configured to transmit sanitizing EO radiation 135, as disclosed herein (e.g., may comprise transparent materials). Alternatively, or in addition, the SEOR components 130 may be disposed within protection members, such as sleeves, tubes, and/or the like, which may be comprised of transparent materials (not shown in FIG. 3G to avoid obscuring details of the illustrated embodiments).

The sanitization device 110 may further comprise a peripheral compartment 340, which may comprise componentry of the sanitization device 110, as disclosed herein (e.g., power module 111, control componentry 112, ES component 136, and/or the like). An end portion 339 of the support member 338 may extend through the second sidewalls 334 into the peripheral compartment 340. The support member 338 may be configured to transmit recovery EO radiation 137 produced by the ES component 136 within the peripheral compartment 340 (and received at the end portion 339 thereof) into the interior compartment 122. The support member 338 and inner surfaces of the interior compartment 122 may be configured to reflect and/or transmit the recovery EO radiation 137 within the interior compartment 122 such that recovery EO radiation 137 is received by substantially all of the SEOR components 130 disposed therein. In some embodiments, the end portion 339 of the support member 338 is configured to refract and/or diffuse EO radiation such that EO radiation received at the end portion 339 is refracted and/or diffused through the support member 338 and into the interior compartment 12. The support member 338 may comprise a quartz plate having an end configured to refract and/or diffuse EO radiation. In some embodiments, the end portion 339 of the support member 338 is configured to receive, refract, and/or diffuse EO radiation (e.g., the end portion 3439 may be rounded, beveled, dimpled, and/or the like). The end portion 339 and/or ES component 136 may be positioned and/or oriented relative to one another within the peripheral compartment 340 in order to, inter alia, facilitate transmission of the recovery EO radiation 137. The end portion 339 and/or ES component 136 may be arranged such that the recovery EO radiation 137 is directed towards the end portion 339, is directed towards the end portion 339 at a particular angle ad/or orientation, and/or the like.

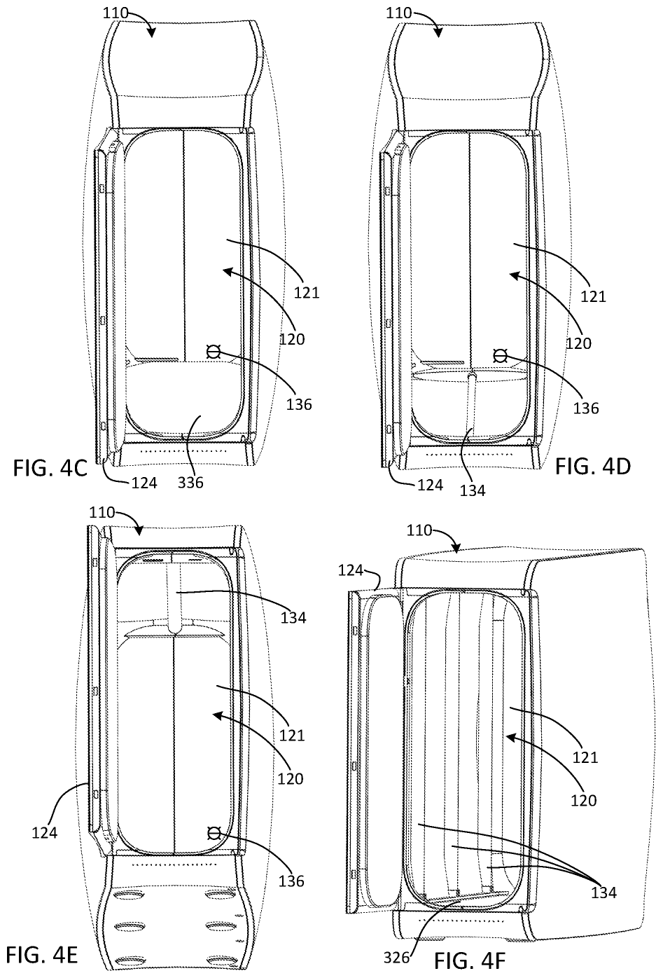

FIGS. 4A-F illustrate further embodiments of a sanitization device 110, as disclosed herein (fourth embodiments having a cabinet configuration). As illustrated in FIG. 4A, the sanitization device 110 may comprise a power module 111 configured to receive power from an external power source 11, control componentry 112 comprising one or more of SD metadata 113, a controller 212, a recovery module 214, a communication interface 118 configured to couple the control componentry 112 to a network 101, and/or the like. The componentry may be disposed within a peripheral compartment 340, as disclosed herein (not shown in FIGS. 4A-4F to avoid obscuring details of the illustrated embodiments). The sanitization device 110 may further comprise a plurality of SEOR components 130 disposed within an interior compartment of the enclosure 120, as illustrated in FIGS. 4B-4F.

As shown in FIGS. 4A and 4B, the access component 124 of the sanitization device 110 may comprise a door configured to provide access to the interior compartment 122 of the enclosure 120. The access component 124 may comprise closure component(s) 126 configured to determine a state of the access component 124 and/or lock the access component 124 in the closed configuration, as disclosed herein. Inner surfaces 121 of the access component 124 and/or interior compartment 122 may be configured to reflect sanitizing EO radiation, as disclosed herein. The sanitization device 110 may further comprise HMI components 114, such as an HMI element 315. The HMI element 315 may be configured to indicate status information pertaining to the sanitization device 110 and/or receive user commands pertaining to operation of the sanitization device 110, as disclosed herein. The sanitization device 110 may further comprise a power module 111 and/or control componentry 112, as disclosed herein (not shown in FIGS. 4A-4F to avoid obscuring details of the illustrated embodiments). As illustrated in FIGS. 4B and 4F, the sanitization device 110 may comprise SEOR component(s) 130, including CCFL 134 disposed along respective sides of the interior compartment 122. The sanitization device 110 may comprise a plurality of CCFL 134 disposed on vertical side(s) of the enclosure 120.

FIG. 4C is a top front perspective view of the disclosed sanitization device 110 in the open configuration. As illustrated, an inner compartment 122 of the sanitization device 110 may comprise a support member 338. The support member 338 may be configured to hold target object(s) 10, as disclosed herein. The support member 338 may be transparent to sanitizing EO radiation 135 emitted by the SEOR components 130 of the sanitization device 110. FIG. 4D is a top front perspective view of the disclosed sanitization device 110 in which the support member 338 is omitted. As shown, the sanitization device 110 may comprise a CCFL 134 disposed along a bottom section of the enclosure 120 (a lower CCFL 134). The support member 338 illustrated in FIG. 4C may be configured to maintain target object(s) above the lower CCFL 134. FIG. 4E is a bottom front perspective view of the disclosed sanitization device in the open configuration. As shown, the sanitization device 110 may further comprise a CCFL 134 disposed along a top section of the enclosure 120 (an upper CCFL 134). The enclosure 120 may further comprise an EO component 136 configured to, inter alia, emit recovery EO radiation 137 within the inner compartment 122. As illustrated, the EO component 136 may be disposed within the interior compartment 122. Alternatively, the EO component 136 may be disposed in a peripheral compartment of the sanitization device 110, and recovery EO radiation 137 may be transmitted into the interior compartment 122 through the support member 138, as disclosed herein.

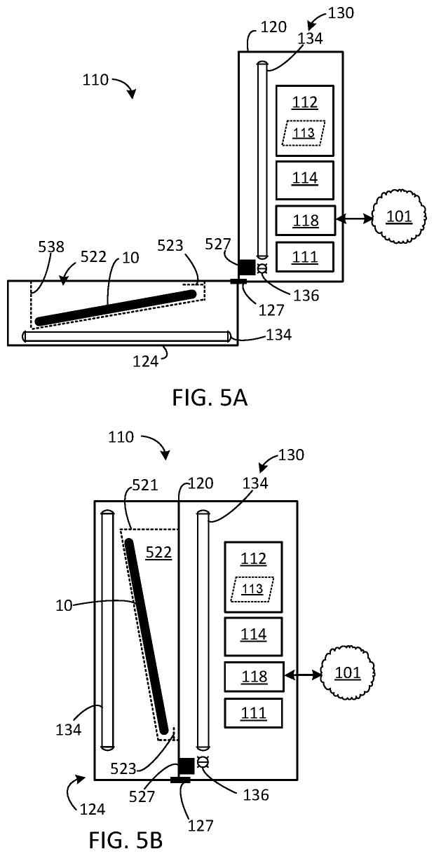

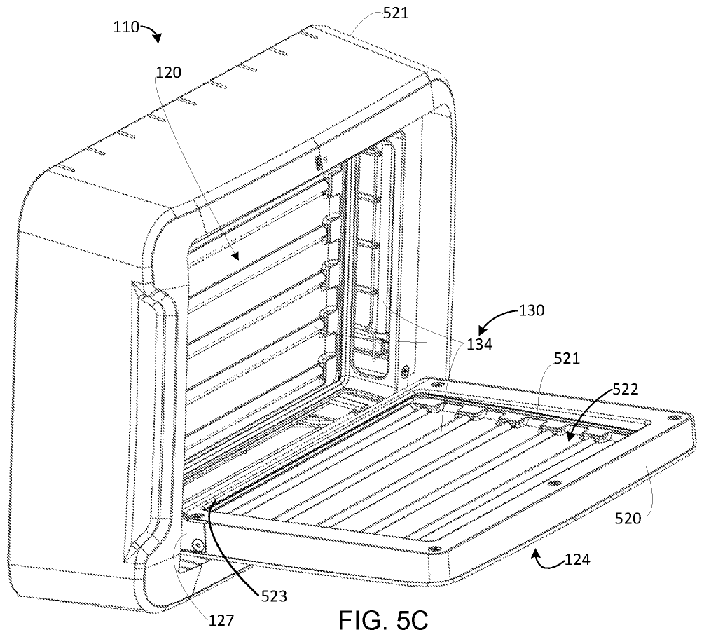

FIGS. 5A-5C illustrate further embodiments of a sanitization device 110, as disclosed herein (fifth embodiments). The sanitization device 110 may comprise a power module 111, control componentry 112, HMI components 114, a network interface 118, a plurality of SEOR components 130, and an EO component 136, as disclosed herein. The sanitization device 110 may further comprise a controller 212, recovery module 214, and/or the like (not shown in FIGS. 5A-5C to avoid obscuring details of the illustrated embodiments).

The sanitization device 110 may further comprise an enclosure 120 configured to receive target object 10. Inner surfaces of the enclosure 120 may be configured to reflect sanitizing EO radiation, as disclosed herein. As illustrated the access component 124 of the sanitization device 110 may comprise a support member 538 which may be configured to receive, hold, and/or secure a target object 10 at a particular orientation and/or position within the enclosure 120. The support member 538 may be configured to maintain the target object 10 in an orientation and/or position configured to allow sanitizing EO radiation 135 emitted to irradiate substantially the entire surface of target object 10. In some embodiments, the support member 538 may be transparent (or substantially transparent). In some embodiments, an SEOR component 130 may be placed below the support member 538 such that sanitizing EO radiation 135 is emitted through the support member 528 onto the surface of the target object 10.

In some embodiments, the support member 538 may comprise a flat support member (e.g., plate) configured to hold the target object in a horizontal orientation. In some embodiments, a connector of the charger may be rigidly attached to the target object 10 such that the target object 10 is secured within the enclosure 120. Alternatively, or in addition, the support member 538 may comprise a textured surface capable of preventing or minimizing movement of the target object 10. In some embodiments, the support member 538 further comprises raised members that prevent the target object 10 from sliding off the support member 538. The raised members may be transparent to the sanitizing EO radiation. In another embodiment, the raised members are reflective to the sanitizing EO radiation.

The HMI components 114 may comprise a hands-free HMI element, which may be configured to selectively open the enclosure 120 to receive a target object 10 and close the enclosure 120 in preparation for performing a sanitization operation in response to hands-free user inputs. As used herein, a “hands-free” input refers to an input that does not require the user to touch the sanitization device 110 and/or the HMI components 114 thereof. The hands-free HMI element of the sanitization device 110 may include, but is not limited to: a motion sensor, a gesture sensor, an acoustic sensor, a camera, an image capture device, a capacitive sensor, a thermal sensor, and/or the like. The hands-free HMI element may be configured to open the enclosure 120 to receive a target object 10 (by use of the actuator 527) in response to a first hands-free input and to perform a sanitization operation in response to a second hands-free input (e.g., close the enclosure 120 and/or activate the emitters 468, as disclosed herein).

In some embodiments, the enclosure 120 may comprise an acoustic conduit, which may comprise an opening and/or exit configured to provide an acoustic path or channel between the interior and exterior of the enclosure 120 (not shown in FIG. 4A to avoid obscuring the details of the illustrated embodiments). Sound emitted by the target object 10 within the enclosure 120 may pass through the acoustic conduit to the exterior of the enclosure 120. The acoustic conduit may, therefore, allow the user to hear alerts or alarms generated by the target object 10 while the target object 10 is within the enclosure 120. The acoustic conduit may be further configured to prevent EO radiation from escaping the enclosure 120. Accordingly, the acoustic conduit may be configured to block and/or prevent optical paths between the interior of the enclosure 120 and the exterior of the enclosure 120. In some embodiments, a shape of the acoustic conduit may be configured to block EO radiation; the acoustic conduit may be curved, tapered, and/or otherwise adapted to prevent EO radiation leakage. Alternatively, or in addition, an outer surface of the acoustic conduit may be composed of materials configured to absorb EO radiation emitted by the emitter 468. The acoustic conduit may be configured such that there is no line-of-sight or optical path from the interior of the enclosure 120 to the exterior of the enclosure 120. In some embodiments, the acoustic conduit comprises a narrow slot leading from the interior of the enclosure 120 to the exterior of the enclosure 120. In some embodiments the acoustic conduit comprises a membrane of EO radiation absorptive material configured to block EO radiation, while readily allowing acoustic signals to pass through. In some embodiments, filaments of EO radiation absorptive material are placed within the acoustic conduit to absorb sanitizing EO radiation while allowing acoustic signals to pass.

In some embodiments, the enclosure 120 and/or acoustic conduit are configured to amplify sounds therein (e.g., amplify acoustic signals generated within the enclosure 120). In some embodiments the acoustic conduit comprises an acoustic megaphone configured to amplify sound or other acoustic signals originating within the enclosure 120. In some embodiments, the acoustic conduit comprises a horn configured to resonate acoustic signals.

In some embodiments, the sanitization device 110 comprises a charger configured to charge or recharge the target object 10 disposed therein (not shown in FIGS. 5A-5C to avoid obscuring the details of the illustrated embodiments). In at least one embodiment, the charger is configured to charge multiple target objects 10. The charger may comprise a connector configured to supply electrical power to the target object 10. In one embodiment, the charger includes a connector for each of a plurality of the target objects 10 that may be placed within the enclosure 120. The connector may be a physical connector that plugs into the target object 10, such as a Universal Serial Bus (USB) connector, mini-USB connector, micro-USB connector, 30-pin connector, proprietary connector, or the like. Alternatively, or in addition, the charger may comprise an inductive coil to transfer power wirelessly to the target object 10. In some embodiments the connector of the charger may be further configured to act as a docking connector for the target object 10 (e.g., communicate data between the target object 10 and a computing device, hub, or the like). The sanitization device 110 may be configured to act as an end node of the data connection or may be configured to act as an intermediary node (hub) used to establish a data connection between the PD and another external computing device. In some embodiments the charger may comprise a removable adaptor capable of connecting to various different types of connectors and/or target objects 10. In some embodiments the connector of the charger is extendable so that the target object 10 can be positioned at different locations and/or orientations within the enclosure 120.

In some embodiments the charger comprises a pass-through port configured to allow a cord or cable of a third-party charger to pass into the enclosure 120. The port and/or opening may be configured to prevent EO radiation from escaping the enclosure 120. Accordingly, the port and/or opening may comprise a gasket, pass-through cable, or other mechanisms and/or structures for blocking EO radiation. Alternatively, the charger may comprise an intermediary cable or cord with an exterior connector for connecting to a third-party charger and an interior connector.

The sanitization device 110 may further comprise a closure component 126 configured to maintain the enclosure 120 in a closed configuration. The mechanisms may be further configured to prevent EO radiation from escaping the enclosure 120. In some embodiments, the enclosure 120 may comprise a pair of magnets configured to secure two halves of the enclosure 120 to one another. In some embodiments, the enclosure 120 may comprise a spring in a hinge that applies a closing force thereto. In some embodiments, the enclosure 120 comprises a bi-stable spring, or other suitable mechanism, where one stable state corresponds to a closed configuration and the other stable state corresponds to an open position. In one embodiment, the enclosure 120 comprises a slide member, such as a slide member on a drawer or cover, that slopes toward a closed position such that a drawer or cover is drawn toward the closed position by gravity.

In some embodiments, the enclosure 120 may comprise an EO radiation seal configured to prevent leakage of EO radiation. The radiation seal may comprise a gasket and/or lips formed at the opening of the enclosure 120. In some embodiments, the EO radiation seal(s) may comprise material configured to absorb EO radiation. Portions of the EO radiation seal(s) may be formed from reflective materials configured to reflect EO radiation back into the enclosure 120.

In some embodiments, the opening of the sanitization device 110 comprises an access component 124 having an inner surface 521. The access component 124 may be coupled to a bottom portion of the enclosure 120 by a hinge 127 or other structure. The actuator 527 may be configured to reposition the access component 124 to thereby open and close the enclosure 120. When in the closed configuration, the access component 124 may be coupled to an outer surface of the enclosure 120 thereby enclosing an interior region of the enclosure 120. When in the open configuration, the access component 124 may rotate away from the enclosure 120 (via bottom hinges 469) thereby exposing the interior of the enclosure 120. The inner surface 521 of the access component 124 may be angled such that a target object 10 placed thereon remains secured on the access component 124 as the access component 124 moves from the open configuration (in which the access component 124 is substantially horizontal) to the closed configuration (in which the access component 124 is substantially vertical). The inner surface 521 of an interior compartment 522 of the enclosure 120 may be angled such that, when in the open configuration, the end of the inner surface 521 closest to the hinge 469 (and the bottom of the enclosure 120) is raised relative to the opposite end of the inner surface 521.

FIGS. 5A and 5B depict embodiments in which the access component 124 rotates via the bottom hinges 469 (or other members) from an open configuration to a closed configuration (and vice versa). As illustrated in FIG. 4B, when in the open configuration, the inner surface 521 of the access component 124 is exposed to receive a target object 10. The inner surface 521 may be angled such that an end proximate to the enclosure 120 is raised relative to the end farther from the enclosure 120. As illustrated in FIG. 4C, the actuator 527 may rotate the access component 124 to the closed configuration (via the bottom hinges 469). In the closed configuration, the target object 10 may be secured within the enclosure 120 due to, inter alia, the angle of the inner surface 521 of the access component 124. The target object 10 may be thereby secured within the enclosure 120 without the need for additional securing members, which may block and/or diffuse EO radiation of the emitters 468 (and prevent the target object 10 from being fully sanitized). The inner surface 521 may be formed of materials that are substantially transparent to the EO radiation emitted by the EO emitters 468. The access component 124 and/or inner surface 521 may comprise one or more notches, grooves, and/or the like to further secure the target object 10 within the enclosure 120. As disclosed above, the actuator 527 may be configured to open and close the access component 124 in response to hands-free inputs received via the HMI components 114 (via the hands-free HMI element). In such embodiments, a user 182 may sanitize a target object 10 without touching the exterior surface of the sanitization device 110. The user 182 may touch the inner surface 521 of the access component 124 while placing the target object 10 therein (and removing the target object 10 after sanitation is complete). In some embodiments, the sanitization device 110 is configured to sanitize the inner surface 521 before opening the access component 124 to ensure that the user 182 and/or target object 10 are not subject to any preexisting contaminants within the enclosure 120. Although FIGS. 4B and 4C illustrate particular embodiments for hands-free operation of a sanitization device 110, the disclosure is not limited in this regard and could be adapted to use any suitable hands-free mechanisms including, but not limited to: a self-actuated tray to slide into and out of the enclosure 120, an insertion slot, a rotating opening, and/or the like.

FIG. 5C depicts another embodiment of the sanitization device 110 disclosed herein. FIG. 5C depicts the sanitization device 110 in the open configuration in which the access component 124 is rotated away from the enclosure 120 via the bottom hinges 469 (and the actuator 527, not shown to avoid obscuring the details of the illustrated embodiments). As illustrated, the inner surface 521 of the access component 124 may be angled such that an end of the inner surface 521 proximate to the hinges 469 is raised relative to the opposite end. Accordingly, a target object 10 placed on the inner surface 521 may remain secured thereon when the sanitization device 110 transitions to the closed configuration (e.g., the access component 124 rotates on the hinges 469 to cover the enclosure 120). The sanitization device 110 of the FIG. 5 embodiment may be configured to be mounted vertically (e.g., on a wall).

Discussion is now given to various example embodiments. An apparatus may comprise an enclosure, a sanitizing element, a stimulus component, one or more sensors, and a control circuit. The enclosure may comprise an inner cavity for receiving a target object. The sanitizing element may be coupled to the inner cavity. The sanitizing element may be configured to convert electrical energy into sanitizing electro-optical radiation (SEOR) and expose the target object within the inner cavity to the SEOR. The stimulus component may be coupled to the inner cavity and exposed to the sanitizing element. The stimulus component may be configured to emit recovery electro-optical radiation to the sanitizing element. The one or more sensors may be configured to sense electrical characteristics of the sanitizing element. The control circuit may be configured to determine, based on the electrical characteristics, that the sanitizing element is in a dormant state. The control circuit may be configured to electrically enable the stimulus component to emit the recovery electro-optical radiation to the sanitizing element.

The apparatus may include additional features or elements. A primary compartment may be within the inner cavity of the enclosure. The primary compartment may include the sanitizing element and is configured to receive the target object. A secondary compartment may be within the inner cavity of the enclosure. The stimulus component may be located at least partially within the secondary compartment. A support member may extend from the secondary compartment into the primary compartment. The support member may be configured to cause placement of the target object within the primary compartment and between at least two walls of the enclosure. The support member may be configured to transmit at least some of the recovery electro-optical radiation produced by the stimulus component into the primary compartment that includes the sanitizing element. The one or more sensors may be configured to detect the sanitizing element electrically as an open circuit or a capacitor. The control circuit may be configured to determine, based on the electrical characteristics of the sanitizing element, a failure of the sanitizing element. The one or more thermal sensors may be configured to determine temperature characteristics of an environment within the enclosure. The control circuit may be configured to modify, based on the temperature characteristics sensed by the one or more thermal sensors, operation of the sanitizing element. The enclosure may comprise an access component that includes a support member configured to secure the target object at a determined orientation or position within the enclosure. The sanitizing element may be a cold cathode fluorescence lamp (CCFL). The dormant state may be a low electron state. The recovery electro-optical radiation may be visible spectrum light.

An apparatus may comprise an enclosure, a sanitizing element and a control circuit. The enclosure may comprise an inner cavity for receiving a target object. The sanitizing element may be coupled to the inner cavity. The sanitizing element may be configured to expose the target object within the inner cavity to sanitizing electro-optical radiation (SEOR) during an active state. The control circuit may be configured to determine, based on electrical characteristics of the sanitizing element, a failure of the sanitizing element.

The apparatus may include additional features or elements. The one or more sensors may be coupled to the sanitizing element and configured to sense the electrical characteristics of the sanitizing element. A stimulus component may be coupled to the inner cavity and exposed to the sanitizing element. The stimulus component may be configured to emit recovery electro-optical radiation to the sanitizing element. The control circuit may be configured to determine, based on the electrical characteristics, that the sanitizing element is in a dormant state. The control circuit may be configured to electrically enable the stimulus component to emit the recovery electro-optical radiation to the sanitizing element. The one or more thermal sensors may be configured to determine temperature characteristics of an environment within the enclosure. The control circuit may be configured to modify, based on temperature characteristics sensed by the one or more thermal sensors, operation of the sanitizing element. The enclosure may comprise an access component that includes a support member configured to secure the target object at a determined orientation or position within the enclosure. A human-machine interface may be mechanically coupled to the enclosure and configured to indicate the failure of the sanitizing element. The sanitizing element may be removably mechanically coupled to the enclosure.

An apparatus may comprise an enclosure, a sanitizing element, one or more thermal sensors, and a control circuit. The enclosure may comprise an inner cavity for receiving a target object. The sanitizing element may be coupled to the inner cavity. The sanitizing element may be configured to expose the target object within the inner cavity to sanitizing electro-optical radiation (SEOR) during an active state. The one or more thermal sensors may be configured to determine temperature characteristics of an environment within the enclosure. The control circuit may be configured to modify, based on the temperature characteristics sensed by the one or more thermal sensors, operation of the sanitizing element.

The apparatus may include additional features or elements. A stimulus component may be coupled to the inner cavity and exposed to the sanitizing element. The stimulus component may be configured to emit recovery electro-optical radiation to the sanitizing element. The control circuit may be configured to determine, based on electrical characteristics of the sanitizing element, that the sanitizing element is in a dormant state. The control circuit may be configured to electrically enable the stimulus component to emit the recovery electro-optical radiation to the sanitizing element. A primary compartment may be within the inner cavity of the enclosure. The primary compartment may include the sanitizing element. A secondary compartment may be within the inner cavity of the enclosure. The stimulus component may be configured to generate the recovery electro-optical radiation within the secondary compartment. A support member may extend from the secondary compartment into the primary compartment. The support member may be configured to transmit at least some of the recovery electro-optical radiation generated by the stimulus component into the primary compartment that includes the sanitizing element. The control circuit may be configured to determine, based on electrical characteristics of the sanitizing element, a failure of the sanitizing element. The enclosure may comprise an access component that includes a support member configured to secure the target object at a determined orientation or position within the enclosure. The environment of the enclosure may be a temperature of the enclosure or a temperature of the sanitizing element. The control circuit may reduce an intensity of the SEOR and/or increase a duration of the active state.

An apparatus may comprise an enclosure, a sanitizing element, a security sensor, a network interface, and a control circuit. The enclosure may comprise an inner cavity for receiving a target object. The sanitizing element may be coupled to the inner cavity. The sanitizing element may be configured to expose the target object within the inner cavity to sanitizing electro-optical radiation (SEOR) during an active state. The security sensor may be configured to sense a security event. The network interface may be configured to transmit messages via a wireless network. The control circuit may be configured to send, based on the security sensor sensing a security event, a message via the wireless network to the management system.

The apparatus may include additional features or elements. The network interface may be a personal area network interface configured to send messages to the management service via a second wireless network as relayed by the target object. The security sensor may be an electrical sensor. The security event may be disconnection from a power source. The security sensor may be a geographic location sensor (e.g., GPS, GLONASS, cellular triangulation, dead reckoning, acceleration, other location determination technologies, any combination thereof, etc.). The security event may be movement beyond a threshold from a determined location. The security sensor may be a geographic location sensor. The security event may be a location of the apparatus outside a geofenced area. The security sensor may be a locking mechanism. The security event may be tampering with the locking mechanism. The security event may be bypassing the locking mechanism. The security event may be disabling the locking mechanism. The security event may be a failure to unlock the locking mechanism. A primary power supply may be configured to provide power to the sanitizing element, security sensor, network interface and control circuit. A portable secondary power source may be configured to provide, based on a disconnection of the primary power supply from a power source, power to the security sensor, network interface and control circuit.

In the disclosure, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with OCR systems, server computers, and/or communications networks have not been shown or described in detail to avoid unnecessarily obscuring descriptions of the embodiments.

Unless the context requires otherwise, as used herein, the word “comprise” and variations thereof, such as “comprises” and “comprising,” are to be construed in an open, inclusive sense, that is as “including, but not limited to.”

As used herein, “one embodiment” or “an embodiment” means that a particular feature, structure, or characteristic described in connection with the embodiment is included in at least one embodiment. Thus, the appearances of the phrases “in one embodiment” or “in an embodiment” herein are not necessarily all referring to the same embodiment. Furthermore, the particular features, structures, or characteristics may be combined or assembled in any suitable manner in one or more embodiments.

As used herein, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The headings and Abstract of the Disclosure provided herein are for convenience only and do not interpret the scope or meaning of the embodiments.

Changes may be made to the details of the above-described embodiments without departing from the underlying principles of the invention. The scope of the present invention should, therefore, be determined only by the following claims.

1. An apparatus, comprising:
an enclosure comprising an inner cavity for receiving a target object;
a sanitizing element disposed within the inner cavity, wherein the sanitizing element is configured to:
convert electrical energy into sanitizing electro-optical radiation (SEOR);
expose the target object within the inner cavity to the SEOR during an active state;
a stimulus component within the inner cavity and exposed to the sanitizing element, wherein the stimulus component is configured to emit recovery electro-optical radiation to the sanitizing element;
one or more sensors configured to sense electrical characteristics of the sanitizing element; and
a control circuit configured to:
determine, based on the electrical characteristics, that the sanitizing element is in a dormant state; and
electrically enable the stimulus component to emit the recovery electro-optical radiation to the sanitizing element.

2. The apparatus of claim 1, further comprising:
a primary compartment within the inner cavity of the enclosure, wherein the primary compartment includes the sanitizing element and is configured to receive the target object;
a secondary compartment within the inner cavity of the enclosure, wherein the stimulus component is located at least partially within the secondary compartment; and
a support member extending from the secondary compartment into the primary compartment, and configured to cause placement of the target object within the primary compartment and between at least two walls of the enclosure,
wherein the support member is configured to distribute at least some of the recovery electro-optical radiation produced by the stimulus component into the primary compartment that includes the sanitizing element.

3. The apparatus of claim 1, wherein to sense the electrical characteristics of the sanitizing element further comprises detecting the sanitizing element electrically as an open circuit or a capacitor.

4. The apparatus of claim 1, wherein the control circuit is further configured to determine, based on the electrical characteristics of the sanitizing element, a failure of the sanitizing element.

5. The apparatus of claim 1, further comprising:
one or more thermal sensors configured to determine temperature characteristics of an environment within the enclosure,
wherein the control circuit is further configured to modify, based on the temperature characteristics sensed by the one or more thermal sensors, operation of the sanitizing element.

6. The apparatus of claim 1, wherein the enclosure further comprises an access component that includes a support member configured to secure the target object at a determined orientation or position within the enclosure.

7. The apparatus of claim 1, wherein the sanitizing element is a cold cathode fluorescence lamp (CCFL) and the dormant state is a low electron state.

8. The apparatus of claim 1, wherein the recovery electro-optical radiation is visible spectrum light.

9. An apparatus, comprising:
an enclosure comprising an inner cavity for receiving a target object;
a sanitizing element within the inner cavity, wherein the sanitizing element is configured to expose the target object within the inner cavity to sanitizing electro-optical radiation (SEOR) during an active state; and
a control circuit configured to determine, based on electrical characteristics of the sanitizing element, a failure of the sanitizing element.

10. The apparatus of claim 9, further comprising one or more sensors coupled to the sanitizing element and configured to sense the electrical characteristics of the sanitizing element.

11. The apparatus of claim 9, further comprising:
a stimulus component coupled to the inner cavity and exposed to the sanitizing element, wherein the stimulus component is configured to emit recovery electro-optical radiation to the sanitizing element,
wherein the control circuit is further configured to:
determine, based on the electrical characteristics, that the sanitizing element is in a dormant state; and
electrically enable the stimulus component to emit the recovery electro-optical radiation to the sanitizing element.

12. The apparatus of claim 9, further comprising:
one or more thermal sensors configured to determine temperature characteristics of an environment within the enclosure,
wherein the control circuit is further configured to modify, based on temperature characteristics sensed by the one or more thermal sensors, operation of the sanitizing element.

13. The apparatus of claim 9, wherein the enclosure further comprises an access component that includes a support member configured to secure the target object at a determined orientation or position within the enclosure.

14. The apparatus of claim 9, further comprising a human-machine interface mechanically coupled to the enclosure and configured to indicate the failure of the sanitizing element.

15. The apparatus of claim 9, wherein the sanitizing element is removably mechanically coupled to the enclosure.

16. An apparatus, comprising:
an enclosure comprising an inner cavity for receiving a target object;
a sanitizing element coupled to the inner cavity, wherein the sanitizing element is configured to expose the target object within the inner cavity to sanitizing electro-optical radiation (SEOR) during an active state;
one or more thermal sensors configured to determine temperature characteristics of an environment within the enclosure; and
a control circuit configured to modify, based on the temperature characteristics sensed by the one or more thermal sensors, operation of the sanitizing element.

17. The apparatus of claim 16, further comprising:
a stimulus component coupled to the inner cavity and exposed to the sanitizing element, wherein the stimulus component is configured to emit recovery electro-optical radiation to the sanitizing element,
wherein the control circuit is further configured to:
determine, based on electrical characteristics of the sanitizing element, that the sanitizing element is in a dormant state; and
electrically enable the stimulus component to emit the recovery electro-optical radiation to the sanitizing element.

18. The apparatus of claim 17, further comprising:
a primary compartment within the inner cavity of the enclosure, wherein the primary compartment includes the sanitizing element;
a secondary compartment within the inner cavity of the enclosure, wherein the stimulus component is configured to generate the recovery electro-optical radiation within the secondary compartment; and
a support member extending from the secondary compartment into the primary compartment and configured to distribute at least some of the recovery electro-optical radiation generated by the stimulus component into the primary compartment that includes the sanitizing element.

19. The apparatus of claim 16, wherein the control circuit is further configured to determine, based on electrical characteristics of the sanitizing element, a failure of the sanitizing element.

20. The apparatus of claim 16, wherein the enclosure further comprises an access component that includes a support member configured to secure the target object at a determined orientation or position within the enclosure.

21. The apparatus of claim 16, wherein the environment of the enclosure is a temperature of the enclosure or a temperature of the sanitizing element.

22. The apparatus of claim 16, wherein to modify the operation of the sanitizing element further comprises to reduce an intensity of the SEOR and increase a duration of the active state.
